(19) United States
(12) Patent Application Publication
(10) Pub. No.: US 2008/0039581 A1
(43) Pub. Date: Feb. 14, 2008

(54) Triarylamine Compounds, Compositions and Devices

(76) Inventors: Beverley Anne Brown, Warrington (GB); Stephen William Leeming, Manchester (GB); Richard Williams, Manchester (GB)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/572,872

(22) PCT Filed: Jul. 21, 2005
(86) PCT No.: PCT/EP05/07943

§ 371 Date: Jan. 29, 2007

(30) Foreign Application Priority Data
Jul. 30, 2004 (GB) 0417036.1

(51) Int. Cl. C07C 211/00; C08F 2/10

(52) U.S. Cl. 524/796; 564/431

(57) ABSTRACT

The invention relates to a compound of Formula (I): wherein: each of R₁, R₂, R₃, R₄, R₅, R₆ and R₇ which may be the same or different on each triarylamine unit of Formula (1) and the same or different from one triarylamine unit of Formula (1) to another is independently hydrogen or an optionally substituted substituent; n is from 5 to 20; and a and b are each independently 0, 1, 2, 3 or 4. Also claimed are compositions comprising a compound of Formula (1) and a synthetic organic polymer resin, a process for preparing a compound of Formula (1), an organic semiconducting layer prepared from the composition, and electronic devices comprising the organic semiconducting layer.

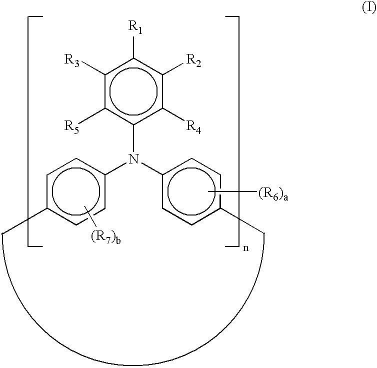

The present invention relates to novel cyclic triarylamine compounds, to compositions comprising cyclic triarylamine compounds and to the use of cyclic triarylamine compounds in electronic devices. The invention also relates to processes for making novel cyclic triarylamines.

In recent years research into organic semiconducting materials has centred around producing versatile, low cost electronic devices. Such materials find application in a wide range of electronic devices and apparatus, including organic field effect transistors (OFETs), organic light emitting diodes (OLEDs), photodetectors, photovoltaic (PV) cells, sensors, memory elements, logic circuits and organic photoconductors (OPCs) in electrophotographic devices.

Improved charge mobility is one goal of new electronic devices. Other goals include stability, solution processability and self organisation of the organic semiconducting layers. It is recognised in the electronics industry that maximisation of these features is essential in the preparation of improved low cost printable electronic devices.

Organic semiconducting materials are known which possess one or two of these required features to an acceptable level, however, to date organic semiconducting materials that possess all four of these features remain illusive.

Polymeric materials that comprise triarylamine repeat units are known in the prior art, as described in WO 99/32537(Avecia), DE 3620649 (BASF) and EP 0765106 (TOYO INK) to name but a few. It is known in the art that when triarylamine units are used in polymeric material the units form amorphous films when incorporated into an electronic device, that is the molecules are not ordered (Materials Research Society Smp. Proc. Vol. 708, 2002).

Triarylamine units have also been incorporated into cyclic compounds, as disclosed in US application number 09/965,589, JP 05323635, and Organic letters 1999, Volume 1, number 13, pages 2053 to 2055 and 2057 to 2060. In these disclosures the triarylamine units are coupled at the meta position and bridged by linking groups. The molecules described in US 09/965,589 are said to provide high luminous efficiency in organic electroluminescent devices.

Surprisingly, it has now been found that certain cyclic molecules based on triaryl amine repeat units possess all four of the desired requirements of organic semi-conductor materials for use in devices for the electronics industry, that is mobility, stability, solution processability and the potential to form ordered films.

Therefore, according to a first aspect of the present invention there is provided a triarylamine compound of Formula (1):

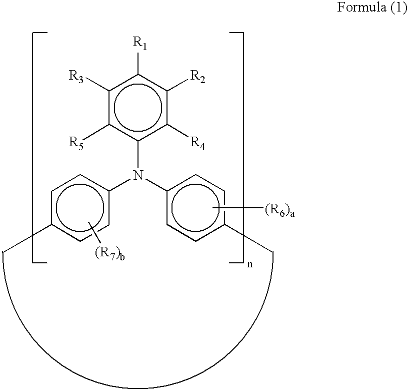

wherein:

each of R₁, R₂, R₃, R₄, R₅, R₆ and R₇ which may be the same or different on each triarylamine unit of Formula (1) and the same or different from one triarylamine unit of Formula (1) to another is independently hydrogen or an optionally substituted substituent;

n is from 5 to 20; and

a and b are each independently 0, 1, 2, 3 or 4.

Preferably, in the triarylamine compounds of Formula (1) each of R₁, R₂, R₃, R₄ and R₅ independently comprises hydrogen or a substituent selected from the group comprising an optionally substituted C₁-C₄₀ carbyl or hydrocarbyl group; an optionally substituted C₁-C₄₀ alkoxy group; an optionally substituted C₆-C₄₀ aryloxy group; an optionally substituted C₇-C₄₀ alkylaryloxy group; an optionally substituted C₂-C₄₀ alkoxycarbonyl group; an optionally substituted C₇-C₄₀ aryloxycarbonyl group; a cyano group (—CN); a carbamoyl group (—C(═O)NH₂); a haloformyl group (—C(═O)—X, wherein X represents a halogen atom); a formyl group (—C(═O)—H); an isocyano group; an isocyanate group; a thiocyanate group; a thioisocyanate group; an optionally substituted amino group; a hydroxy group; a nitro group; a CF₃ group; a halo group; an optionally substituted silyl group; and

each of R₆ and R₇ is independently selected from the group comprising H, CH₃, F, CN or CF₃.

In the cyclic triarylamine compounds of the present invention, the C₁-C₄₀ carbyl or hydrocarbyl may be a saturated or unsaturated acyclic group, or a saturated or unsaturated cyclic group. The C₁-C₄₀ carbyl or hydrocarbyl group includes a C₁-C₄₀ alkyl group, a C₂-C₄₀ alkenyl group, a C₂-C₄₀ alkynyl group, a C₃-C₄₀ allyl group, a C₄-C₄₀ alkyldienyl group, a C₄-C₄₀ polyenyl group, a C₆-C₁₈ aryl or heterocyclic group, a C₆-C₄₀ aralkyl group, a C₆-C₄₀ alkylaryl group, a C₄-C₄₀ cycloalkyl group, a C₄-C₄₀ cycloalkenyl group, and the like. Preferred among the foregoing groups are a C₁-C₂₀ alkyl group, a C₂-C₂₀ alkenyl group, a C₂-C₂₀ alkynyl group, a C₃-C₂₀ allyl group, a C₄-C₂₀ alkyldienyl group, a C₆-C₁₂ aryl group and a C₄-C₂₀ polyenyl group, respectively; more preferred are a C₁-C₁₂ alkyl group, C₂-C₁₀ alkenyl group, a C₂-C₁₀ alkynyl group, a C₃-C₁₀ allyl group, a C₄-C₁₀ alkyldienyl group, a C₆-C₁₂ aryl group and a C₄-C₁₀ polyenyl group respectively.

Examples of the alkyl group include, without limitation, methyl, ethyl, propyl, n-butyl, t-butyl, dodecanyl, trifluoromethyl, perfluoro-n-butyl, 2,2,2-trifluoroethyl, benzyl, 2-phenoxyethyl, etc. Examples of the alkynyl group are ethynyl and propynyl. Examples of the aryl group are, without limitation, phenyl, 2-tolyl, 4-tolyl, naphthyl, biphenyl, 4-phenoxyphenyl, 4-fluorophenyl, 3-carbomethoxyphenyl, 4-carbomethoxyphenyl, etc. Examples of the alkoxy group are, without limitation, methoxy, ethoxy, 2-methoxyethoxy, t-butoxy, etc. Examples of the aryloxy group are, without limitation, phenoxy, naphthoxy, phenylphenoxy, 4-methylphenoxy, etc. Examples of the amino group are, without limitation, dimethylamino, methylamino, methylphenylamino, phenylamino, etc.

In the cyclic triarylamine compounds of Formula (1), the optional substituents on the C₁-C₄₀ carbyl or hydrocarbyl groups for R₁, R₂, R₃, R₄ or R₅ are preferably selected from: silyl, sulpho, sulphonyl, formyl, amino, imino, nitrilo, mercapto, cyano, nitro, halo, C₁₋₄ alkyl, C₆₋₁₂ aryl, C₁₋₄ alkoxy, hydroxy and/or all chemical possible combinations thereof.

Most preferably in the cyclic triarylamine compounds of the present invention R₁, R₂, R₃, R₄ or R₅ are each independently an acyclic group, preferably a saturated acyclic group for example an optionally substituted C₁₋₄₀ alkyl group, more preferably an optionally substituted C₁₋₂₀ alkyl group, most preferably an optionally substituted C₁₋₁₂ alkyl group which is either linear or branched.

Most preferably in the cyclic triarylamine compounds of Formula (1) R₆ and R₇ are each independently H or methyl.

In the compounds of Formula (1), n is an integer of value from 5 to 20. More preferably n is 6 to 15. Furthermore, in the compounds of Formula (1) a and b are integers of independent value 0, 1, 2, 3 or 4.

An example of a preferred cyclic triarylamine compound according to the present invention but not limited thereto is when n comprises a value=6. That is, the triarylamine units of Formula (1) form a cyclic compound which is a hexamer as illustrated in Formula (2):

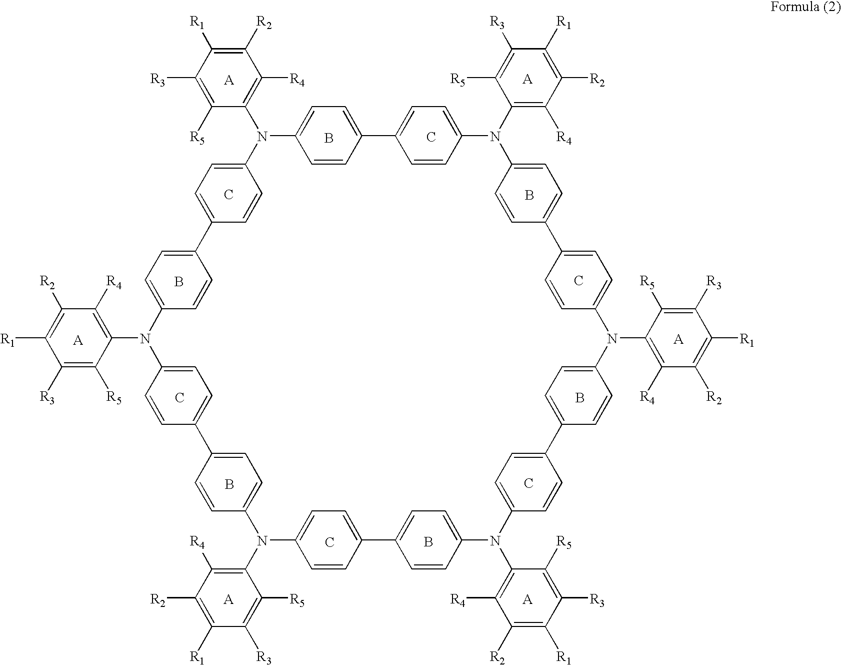

In the hexamer compound of Formula (2), R₁, R₂, R₃, R₄ and R₅ are preferably as described above, a and b are both 4, and R₆ and R₇ are both hydrogen.

In a further example of a hexamer compound of Formula (2), a and b are 1, 2, 3 or 4 and the aryl rings B and C of the triarylamine units are substituted, preferably by methyl, and R₁, R₂, R₃, R₄ and R₅ are as described above.

Whilst not wishing to be bound by any particular theory, the inventors believe that the compounds of the present invention may exhibit the desired properties of improved charge mobility, stability, solution processability and also possess the potential to form ordered films, due to the ability of the compounds to close pack into highly ordered forms. A proposed molecular model illustration of the close packing form of cyclic triarylamine compounds comprising six triarylamine units is shown in FIGS. (1a) and (1b).

It is proposed that the cyclic triarylamine hexamer compounds illustrated by the molecular models in FIGS. (1a) and (1b) above are able to form “chair” like structures similar to that known in the art for cyclohexane. It is proposed that these ‘chair’ like structures are able to align or form ordered films.

It will be realised that the present invention covers all isomeric forms of the compounds described in relation to the first aspect of the present invention.

In a preferred embodiment of the present invention the cyclic triarylamine compounds have a field effect mobility, μ, of more than 10⁻⁵cm²V⁻¹s⁻¹, preferably more than 10⁴cm²V⁻¹s⁻¹, more preferably more than 10⁻³cm²V⁻¹s⁻¹, still more preferably more than 10⁻²cm²V⁻¹s⁻¹ and most preferably more than 10⁻¹cm²V⁻¹s⁻¹.

According to a second aspect of the present invention there is provided a process for making the cyclic triarylamine compounds according to the first aspect of the present invention. A suitable method comprises the following steps:
(i) Preparing a triarylamine monomer (substituted as required) (Formula (3)) using for example either Ullmann or Buchwald reaction conditions from a suitably substituted aniline or secondary amine and a suitably substituted aryl halide (bromide or iodide).

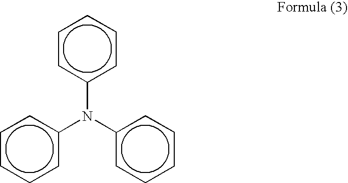

(ii) Brominating available para positions on the triarylamine monomer using for example N-bromosuccinimide (NBS), (Formula (4)).

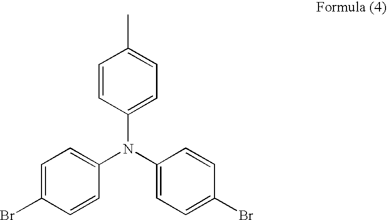

(iii) Converting the brominated compound of Formula (4) to a diboronic ester, via a lithiation step (using for example n-butyl lithium) followed by quenching with an isopropoxy boronic ester, (Formula (5)).

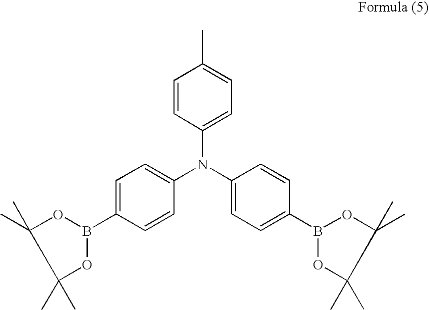

(iv) Synthesising a secondary amine from a suitably substituted aniline derivative and a suitably substituted aryl halide (usually bromide) using for example Buchwald reaction conditions, (Formula (6)).

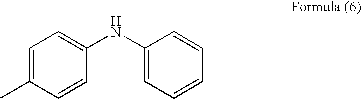

(v) Brominating the secondary amine in the available para position using for example NBS, (Formula (7)).

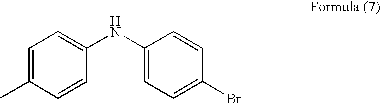

(vi) Protecting the mono-brominated secondary amine (7) with a protecting group (for example ^{t}butoxy carbonyl (Boc), using for example di-^{t}butyl dicarbonate and an acylation catalyst (Formula (8)).

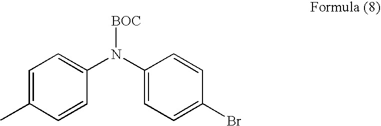

(vii) Coupling together compounds of Formula (5) and (8) using for example a Suzuki coupling reaction to give a ‘trimer’ where both ends of the molecule are protected for example by Boc, (Formula (9)).

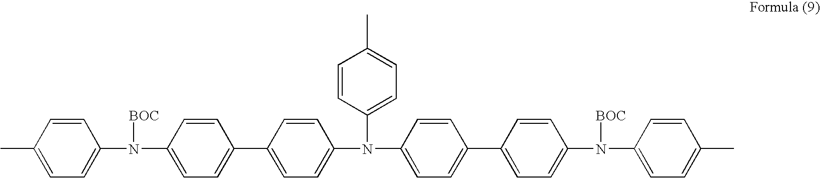

(viii) Removing of the (Boc) protecting groups from the ‘trimer’ using for example trifluoroacetic acid to give a diamine, (Formula (10)).

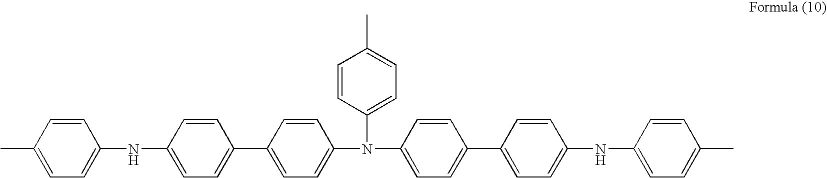

(ix) Cyclising the diamine (Formula (10)) with 4,4′-diiodobiphenyl using for example Ullmann reaction conditions, under high dilution, to yield a novel cyclic triarylamine compound according to the present invention, for example (Compound (9)).

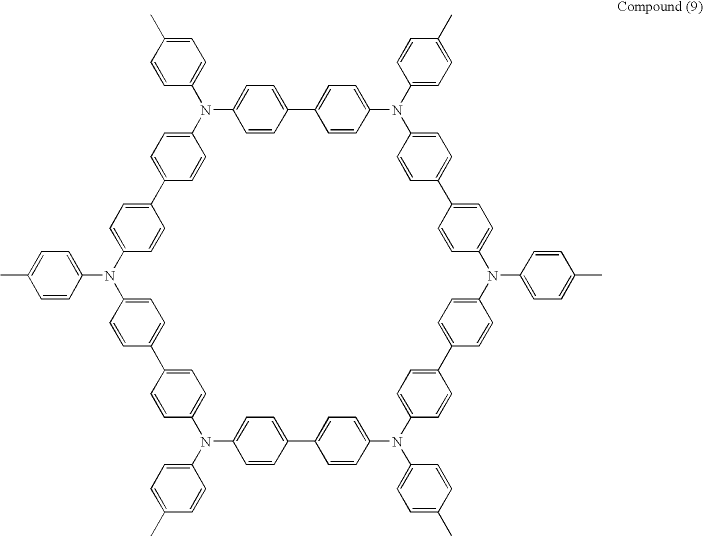

### Alternatively the cyclic triarylamine compounds of the present invention can be prepared using points (i) to (vii) outlined above followed by steps (x) to (xiii).

(x) Preparing 4-Bromo-4′-(trimethylsilyl)biphenyl (Formula (11)) via for example a Suzuki coupling reaction between 1-bromo-4-iodobenzene and 4-(trimethylsilyl)phenyl boronic acid.

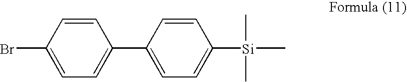

(xi) Preparing a triarylamine trimer of (Formula (12)) from the reaction of 4-bromo-4′-(trimethylsilyl)biphenyl (Formula (11)) and a suitably substituted bis amine trimer (see step (viii) above).

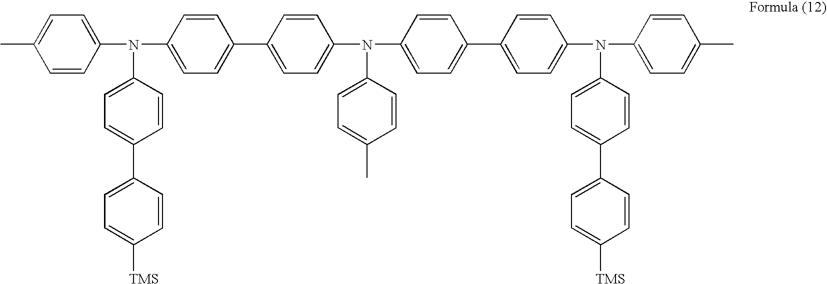

(xii) Preparing a diiodo functionalised “monomer” (Formula (13)) by for example an exchange reaction between a trimethylsilyl group and iodine using iodine monochloride.

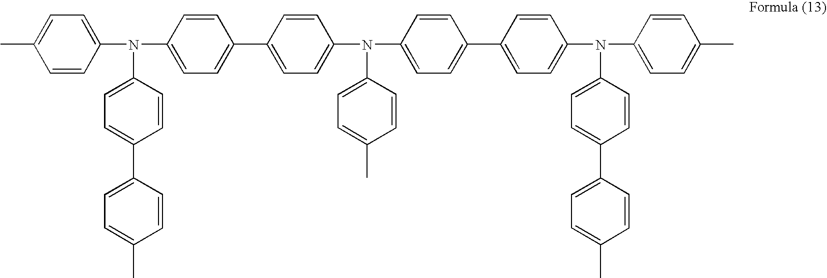

(xiii) Cyclising the diiodo “monomer” of step (xii) with a suitably substituted bis amine “monomer” using for example Buchwald conditions to yield a cyclic triarylamine compound according to the present invention and as illustrated for example by Compound (9) above.

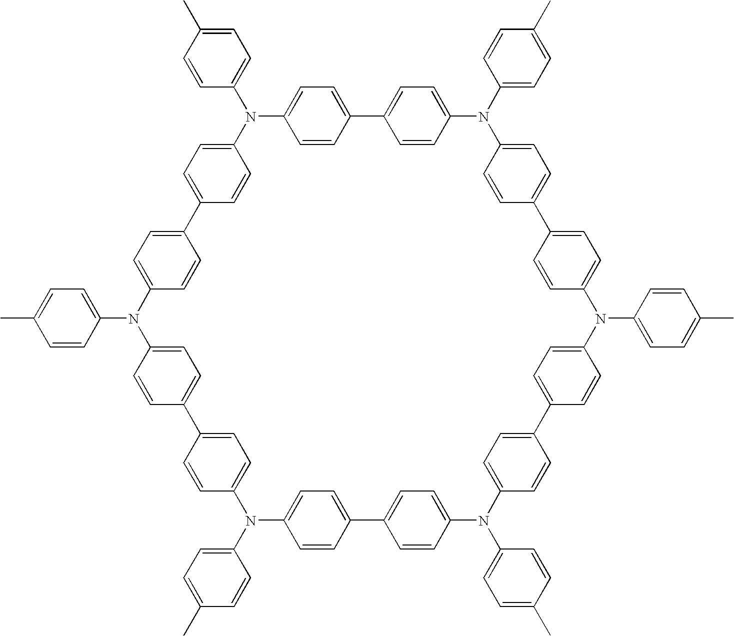

It will be appreciated to the man skilled in the art that the order in which steps (i) to (iii) and (iv) to (vi) are performed prior to steps (vii) to (ix) can be varied. That is, steps (i) to (iii) can be performed either before or after steps (iv) to (vi).

The present invention also relates to compositions comprising compounds of Formula (1). One or more compounds of Formula (1) may be mixed with a synthetic organic polymer resin in order to improve the properties of the composition.

Therefore, according to a third aspect of the present invention there is provided a composition comprising:
(i) a cyclic triarylamine compound of Formula (1),

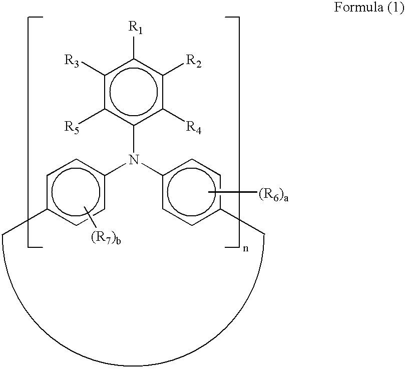

wherein:

each of R₁, R₂, R₃, R₄, R₅, R₆ and R₇, n, a and b are as previously described; and
(ii) a synthetic organic polymer resin.

The proportions of resin to cyclic triarylamine in the composition preferably comprises:1:99 to 99:1, preferably 20:1 to 1:20, more preferably 10:1 to 1:10 even more preferably 5:1 to 1:5, still more preferably 3:1 to 1:3 and most preferably 2:1 to 1:2 (for example 1:1).

The synthetic organic polymer resin includes for example, a thermoplastic polymer, a thermosetting polymer, engineering plastics and the like. The synthetic organic polymer resin may also be a copolymer. Examples of the thermoplastic polymer include: a polyolefin, such as for example polyethylene, polypropylene, polycycloolefin, ethylene-propylene copolymer, etc., polyvinyl chloride, polyvinylidene chloride, polyvinyl acetate, polyacrylic acid, polymethacrylic acid, polystyrene, polyamide, polyester, polycarbonate, etc. Examples of the thermosetting polymer include for example, a phenol resin, a urea resin, a melamine resin, an alkyd resin, an unsaturated polyester resin, an epoxy resin, a silicone resin, a polyurethane resin, etc. Examples of the engineering plastics include for example polyimide, polyphenylene oxide, polysulfone, etc. The synthetic organic polymer resin may also be a synthetic rubber such as for example styrene-butadiene, etc., or a fluoro resin such as for example polytetrafluoroethylene, etc.

The composition according to the third aspect of the present invention may further contain a variety of additives. Examples of the additives include a plasticizer, an antistatic agent, a colorant, a dopant, surfactant etc. Furthermore, the composition may also contain a reinforcing material such as glass fibres, carbon fibres, aramid fibres, boron fibres or carbon nanotubes.

The composition may be prepared into the form of for example fibres, films or sheets, using methods known to one skilled in the art. These methods include, but are not limited thereto, melt spinning, spinning from a solution, dry jet wet spinning, extrusion, flow casting and molding techniques. The fibres, films or sheets may further be processed by roll molding, embossing, postforming or other methods known to one skilled in the art.

Surprisingly and beneficially, it has been found that combining the cyclic triarylamine compounds of the present invention, especially cyclic triarylamine compounds wherein n is 6 (hereinafter referred to as “the cyclic triarylamine”) with a synthetic organic polymer resin (hereinafter simply referred to as “resin”), which serves as organic binder, results in little or no reduction in charge mobility of the cyclic triarylamine. For instance, the cyclic triarylamine may be dissolved in a resin (for example a polystyrene such as α-methyl styrene) and deposited (for example by spin coating), to form an organic semiconducting layer yielding a charge mobility of greater than 10⁻⁴ cm²V⁻¹S⁻¹.

With the use of a resin in the composition of the present invention it has been found that the composition may be coated onto a large area in a highly uniform manner. The use of a resin also enables the cyclic triarylamines to be spin coated onto large areas and still obtain uniform films. Furthermore, it is possible to control the properties of the composition for example, viscosity, solid content, surface tension to enable successful printing to take place. It is also anticipated that the resin fills in volume between the crystalline grains of the organic material, making the organic semiconducting layer less sensitive to air and moisture. For example, layers formed according to the present invention show very good stability in OFET devices in air.

As highlighted above the resin utilised in the composition according to the third aspect of the present invention is a synthetic organic polymer. Such polymers are also often referred to as organic binders or simply binders, which terminology may also be used herein. Preferred resins are materials of low permittivity, that is, those having a permittivity, ε, at 1,000 Hz of no higher than 3.3. The organic resin preferably has a permittivity at 1,000 Hz of less than 3.0, more preferably 2.8 or less. Preferably the synthetic organic polymer resin has a permittivity at 1,000 Hz greater than 1.7. An example of a suitable resin is polystyrene. Further examples are given below in table 1.

In one type of preferred embodiment, the resin is one in which at least 95%, more preferably at least 98% and especially all of the atoms consist of hydrogen, fluorine and carbon atoms.

It is preferred that the resin normally contains conjugated bonds especially conjugated double bonds and/or aromatic rings.

The resin or binder should preferably be capable of forming a film, more preferably a flexible film. Copolymers of styrene and alpha methyl styrene, for example copolymers of styrene, alpha methyl styrene and butadiene may suitably be used.

Resins of low permittivity of use in the present invention have few permanent dipoles which could otherwise lead to random fluctuations in molecular site energies. The permittivity (dielectric constant) can be determined by the ASTM D150 test method.

It is desirable that the permittivity of the resin has little dependence on frequency. This is typical of non-polar materials. Polymers and/or copolymers can be chosen as the resin by the permittivity of their substituent groups. A list of low polarity resins suitable for use in the present invention is given in Table 1 but not limited thereto.

TABLE 1

| | Typical low frequency |
|---|---|
| Resin | permittivity ε |
| Polystyrene | 2.5 |
| poly(α-methyl styrene) | 2.6 |
| poly(α-vinylnaphtalene) | 2.6 |
| poly(vinyltoluene) | 2.6 |
| polyethylene | 2.2-2.3 |
| cis-polybutadiene | 2.0 |
| polypropylene | 2.2 |
| polyisoprene | 2.3 |
| poly(4-methyl-1-pentene) | 2.1 |
| poly (tetrafluoroethylene) | 2.1 |
| poly(chorotrifluoroethylene) | 2.3-2.8 |
| poly(2-methyl-1,3-butadiene) | 2.4 |
| poly(p-xylylene) | 2.6 |
| poly(α-α-α′-α′ tetrafluoro-p-xylylene) | 2.4 |
| poly[1,1-(2-methyl propane)bis(4- | 2.3 |
| phenyl)carbonate] | |
| poly(cyclohexyl methacrylate) | 2.5 |
| poly(chlorostyrene) | 2.6 |
| poly(2,6-dimethyl-1,4-phenylene ether) | 2.6 |
| polyisobutylene | 2.2 |
| poly(vinyl cyclohexane) | 2.2 |

Other polymers suitable for use as resins include: poly(4-methyl styrene), poly(1,3-butadiene) or polyphenylene. Copolymers containing the repeat units of the above polymers are also suitable as resins. Copolymers offer the possibility of improving compatibility with the cyclic triarylamine compounds, modifying the morphology and/or the glass transition temperature of the final composition. It will be appreciated that in the above table certain materials are insoluble in commonly used solvents for preparing the composition. In these cases analogues can be used as copolymers. Some examples of copolymers are given in Table 2 (without limiting to these examples). Both random or block copolymers can also be used. It is also possible to add some more polar monomer components as long as the overall composition remains low in polarity.

TABLE 2

| | typical low |
|---|---|
| Copolymers | frequency permittivity (ε) |
| poly(ethylene/tetrafluoroethylene) | 2.6 |
| poly(ethylene/chlorotrifluoroethylene) | 2.3 |
| fluorinated ethylene/propylene copolymer | 2-2.5 |
| polystyrene-co-α-methyl styrene | 2.5-2.6 |
| ethylene/ethyl acrylate copolymer | 2.8 |
| poly(styrene/10% butadiene) | 2.6 |
| poly(styrene/15% butadiene) | 2.6 |
| poly(styrene/2,4 dimethyl styrene) | 2.5 |

Other copolymers may include: branched or non-branched polystyrene-block-polybutadiene, polystyrene-block(polyethylene-ran-butylene)-block-polystyrene, polystyrene-block-polybutadiene-block-polystyrene, polystyrene-(ethylene-propylene)-diblock-copolymers (For example KRATON®-G1701E, Shell), poly(propylene-co-ethylene) and poly(styrene-co-methylmethacrylate).

The resin itself may be a semiconductor, where it will be referred to herein as a semiconducting binder. The semiconducting binder is still preferably a binder of low permittivity as herein before defined. The semiconducting binder preferably has a number average molecular weight (Mₙ) of between 1×10³ and 1×10⁶, more preferably at least 3000, even more preferably at least 5000. The semiconducting binder preferably has a charge carrier mobility, μ, of at least 10⁻⁴cm²V⁻¹s⁻¹, more preferably at least 10⁻⁴cm²V⁻¹s⁻¹.

In the composition of the present invention there may be used one or more cyclic triarylamine compounds of Formula (1) according to the first aspect of the present invention which, may when there is more than one be the same or different and used either alone or in combination with a synthetic organic polymer resin. Additionally or alternatively, in the composition there may be used two or more synthetic organic polymer resins which may be the same or different as described above.

The resin may be formed in situ by mixing or dissolving the cyclic triarylamine in a precursor of the resin, for example a liquid monomer, oligomer or crosslinkable polymer, optionally in the presence of a solvent, and depositing the mixture or solution, for example by dipping, spray coating, drop cast coating, spraying, painting or printing it onto a substrate to form a liquid layer and then curing the liquid monomer, oligomer or crosslinkable polymer, for example by exposure to radiation, heat or electron beams, to produce a solid layer.

If a preformed resin is used it may be dissolved together with the cyclic triarylamine in a suitable solvent, and the solution deposited for example by dipping, spraying, painting or printing it on a substrate to form a liquid layer and then removing the solvent to leave a solid layer. Suitable solvents are chosen from those classes which are a good solvent for both the resin and cyclic triarylamine, and which upon evaporation from the solution composition give a coherent defect free layer. Suitable solvents for the resin or cyclic triarylamine can be determined by preparing a contour diagram for the material as described in ASTM Method D 3132 at the concentration at which the mixture will be employed. The material is added to a wide variety of solvents as described in the ASTM method. Examples of organic solvents which may be considered include: CH₂Cl₂, CHCl₃, monochlorobenzene, o-dichlorobenzene, tetrahydrofuran, anisole, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, dimethylformamide, dimethylacetamide, dimethylsulfoxide, tetralin, decalin and/or mixtures thereof. After the appropriate mixing and ageing, compositions are evaluated as one of the following categories: complete solution, borderline solution or insoluble. The contour line is drawn to outline the solubility parameter-hydrogen bonding limits dividing solubility and insolubility. ‘Complete’ solvents falling within the solubility area can be chosen from literature values such as published in “Crowley, J. D., Teague, G. S. Jr and Lowe, J. W. Jr., Journal of Paint Technology, 38, No 496, 296 (1966)”. Solvent blends may also be used and can be identified as described in “Solvents, W. H. Ellis, Federation of Societies for Coatings Technology, p9-10, 1986”. Such a procedure may lead to a blend of ‘non’ solvents that will dissolve both the resin and cyclic triarylamine although it is desirable to have at least one true solvent in a blend.

The composition according to the third aspect of the present invention may be prepared by a process which comprises first mixing together both the cyclic triarylamine compound and the resin. Preferably the mixing comprises mixing the two components together in a solvent or solvent mixture. The solvent may be a single solvent or the cyclic triarylamine compound and the organic resin may each be dissolved in a separate solvent followed by mixing the two resultant solutions together to mix the components. The solvent(s) containing the cyclic triarylamine compound and the organic resin may then be applied to a substrate. The solvent(s) may be evaporated to form a layer.

As mentioned above, the aim of the present invention is to achieve efficient electronic devices produced using an organic semiconducting compound which has improved stability and integrity, charge mobility, and the potential to form ordered films which can be solution processed to form an organic semiconducting layer in an electronic device. Layer stability and integrity in devices is achieved by formulating the organic semiconductor in compositions which can be solution processed without destroying the essential characteristics of the organic semiconductors which make it desirable for use in an electronic device.

Therefore according to a fourth aspect of the present invention there is provided an organic semiconducting layer which comprises the composition according to the third aspect of the present invention. Patterning of the semiconducting layer prepared from the composition according to the present invention may be carried out by photolithography or electron beam lithography.

Liquid coating of organic electronic devices such as field effect transistors is more desirable than vacuum deposition techniques. The cyclic triarylamine (and resin compositions) of the present invention enable the use of a number of liquid coating techniques. The organic semiconductor layer may be incorporated into the final device structure by, for example and without limitation, dip coating, spin coating, ink jet printing, letter-press printing, screen printing, doctor blade coating; roller printing, reverse-roller printing; offset lithography printing, flexographic printing, web printing, spray coating, brush coating or pad printing.

Selected cyclic triarylamine and resin compositions of the present invention may be applied to prefabricated device substrates by ink jet printing or microdispensing. Preferably industrial piezoelectric print heads such as but not limited to those supplied by Aprion, Hitachi-Koki, InkJet Technology, On Target Technology, Picojet, Spectra, Trident, Xaar may be used to apply the organic semiconductor layer to a substrate. Additionally semi-industrial heads such as those manufactured by Brother, Epson, Konica, Seiko Instruments Toshiba TEC or single nozzle microdispensers such as those produced by Microdrop and Microfab may be used.

In order to be applied by ink jet printing or microdispensing, the cyclic triarylamine and resin compositions must first be dissolved in a suitable solvent. Solvents must fulfil the requirements stated above and must not have any detrimental effect on the chosen print head. Additionally, solvents should have boiling points greater than 100° C., preferably greater than 140° C. and more preferably greater than 150° C. in order to prevent operability problems caused by the solution drying out inside the print head. Suitable solvents include substituted and non-substituted xylene derivatives, di-C₁₋₂-alkyl formamide, substituted and non-substituted anisoles and other phenol-ether derivatives, substituted heterocycles such as substituted pyridines, pyrazines, pyrimidines, pyrrolidinones, substituted and non-substituted N,N-di-C₁₋₂-alkylanilines and other fluorinated or chlorinated aromatics.

A preferred solvent for depositing the cyclic triarylamine and resin composition by ink jet printing comprises a benzene derivative which has a benzene ring substituted by one or more substituents wherein the total number of carbon atoms among the one or more substituents is at least three. For example, the benzene derivative may be substituted with a propyl group or three methyl groups, in either case there being at least three carbon atoms in total in the substituents. Such a solvent enables an ink jet fluid to be formed comprising solvent, resin and cyclic triarylamine which reduces or prevents clogging of the jets and separation of the components during spraying. The solvent(s) may include those selected from the following list of examples: dodecylbenzene; 1-Methyl-4-tert-butylbenzene; Terpineol; Limonene; Isodurene; Terpinolene; Cymene; Diethylbenzene. The solvent may be a solvent mixture, that is a combination of two or more solvents, each solvent preferably having a boiling point greater than 100° C., more preferably greater than 140° C. Such solvent(s) also enhance film formation in the layer deposited and reduce defects in the layer.

The ink jet fluid (that is, the mixture of solvent, cyclic triarylamine and resin) preferably has a viscosity at 20° C. of 1-100 mPa.s, more preferably 1-50 mPa.s and most preferably 1-30 mPa.s

The use of the resin in the present invention also allows the viscosity of the coating solution to be tuned to meet the requirements of the particular print head.

The semiconducting layer of the present invention is typically at most 1 micron (=1 μm) thick, although it may be thicker if required. The exact thickness of the layer will depend, for example, upon the requirements of the electronic device in which the layer is used. For use in an OFET or OLED, the layer thickness may typically be 500 nm or less.

In the semiconducting layer according to the fourth aspect of the present invention there may be used one or more different cyclic triarylamine compounds of Formula (1) according to the first aspect of the present invention alone or in combination with a resin. Additionally or alternatively, in the semiconducting layer there may be used two or more organic resins as described above in combination with one or more optionally different cyclic triarylamine compounds.

In a fifth aspect of the present invention there is further provided a process for preparing the organic semiconducting layer which comprises (i) depositing on a substrate a liquid layer of a composition which comprises the cyclic triarylamine compound, the organic resin or precursor thereof and optionally a solvent, and (ii) forming from the liquid layer a solid layer which is the organic semiconducting layer.

The proportions of the resin to the cyclic triarylamine in the composition or layer according to the present invention are typically 1:99 to 99:1, preferably 20:1 to 1:20, more preferably 10:1 to 1:10 even more preferably 5:1 to 1:5, still more preferably 3:1 to 1:3 and most preferably 2:1 to 1:2 (for example 1:1).

In the process, the solid layer may be formed by evaporation of the solvent and/or by reacting the resin precursor (if present) to form the resin in situ. The substrate may include any underlying device layer, electrode or separate substrate such as silicon wafer or polymer substrate for example.

In one particular embodiment of the present invention, the resin may be alignable, for example capable of forming a liquid crystalline phase. In that case the resin may assist alignment of the cyclic triarylamine, for example such that the cyclic triarylamine stacks (as proposed and illustrated in FIGS. (1a) and (1b)) are preferentially aligned along the direction of charge transport. Suitable processes for aligning the resin include those processes used to align polymeric organic semiconductors such as described in WO 03/007397 (Plastic Logic).

It is desirable to generate small structures in modern microelectronics to reduce cost (more devices/unit area), and power consumption.

The present invention also provides the use of the semiconducting composition or semiconducting layer in an electronic device. The composition may be used as a high mobility semiconducting material in various devices and apparatus. The composition may be used, for example, in the form of a semiconducting layer or film. For various electronic device applications, the thickness may be less than about one micron thick. The layer may be deposited, for example on a part of an electronic device, by any of the aforementioned solution coating or printing techniques.

The composition may be used, for example as a layer or film, in a field effect transistor (FET) for example as the semiconducting channel, organic light emitting diode (OLED) for example as a hole or electron injection or transport layer or electroluminescent layer, photodetector, chemical detector, photovoltaic cell (PVs), capacitor sensor, logic circuit, display, memory device and the like. The composition may also be used in electrophotographic (EP) apparatus, for example as the organic photoconductor. The composition is preferably solution coated to form a layer or film in the aforementioned devices or apparatus to provide advantages in cost and versatility of manufacture. The improved properties of the composition of the present invention enables such devices or apparatus to operate faster and/or more efficiently. The composition and layer of the present invention are especially suitable for use in an OFET as the semiconducting channel. Accordingly, the invention also provides an organic field effect transistor (OFET) comprising a source electrode, a drain electrode, a gate electrode, dielectric and an organic semiconducting channel connecting the source and drain electrodes, wherein the organic semiconducting channel comprises a layer according to the present invention. Other features of the OFET are well known to those skilled in the art.

Therefore, according to a sixth aspect of the present invention there is provided an electronic device comprising the organic semiconducting layer according to the fourth aspect of the present invention. The electronic device may include, without limitation, an organic field effect transistor (OFET), organic light emitting diode (OLED), photodetector, sensor, logic circuit, memory element, capacitor or photovoltaic (PV) cell. For example, the active semiconductor channel between the drain and source in an OFET may comprise the layer of the invention. As another example, a charge (hole or electron) injection or transport layer in an OLED device may comprise the layer of the invention. The organic semiconducting layer may also find use forming at least part of an organic photoconductor (OPC) in an electrophotographic device. The composition of the present invention and layers formed therefrom have particular utility in semiconductor applications.

An OFET device according to the present invention preferably comprises:
a source electrode,
a drain electrode,
a gate electrode,
a semiconducting layer,
one or more gate insulator layers,
optionally a substrate,
   wherein the semiconducting layer preferably comprises a compound of formula I, or a composition comprising a compound of formula I and an organic resin or binder as described above and below.

The OFET device can be a top gate device or a bottom gate device. Suitable structures and manufacturing methods of an OFET device are known to the skilled in the art and are described in the literature, for example in WO 03/052841.

The gate insulator layer preferably comprises a fluoropolymer, like e.g. the commercially available Cytop 809M® or Cytop 107M® (from Asahi Glass). Preferably the gate insulator layer is deposited, e.g. by spin-coating, doctor blading, wire bar coating, spray or dip coating or other known methods, from a formulation comprising an insulator material and one or more solvents with one or more fluoro atoms (fluorosolvents), preferably a perfluorosolvent. A suitable perfluorosolvent is e.g. FC75® (available from Acros, catalogue number 12380). Other suitable fluoropolymers and fluorosolvents are known in prior art, like for example the perfluoropolymers Teflon AF® 1600 or 2400 (from DuPont) or Fluoropel® (from Cytonix) or the perfluorosolvent FC 43® (Acros, No.12377).

The invention will now be further illustrated by the following examples in which all parts and percentages are by weight unless stated otherwise.

### PREPARATIVE EXAMPLES

### Example 1

### Synthesis of Cyclo-Hexa(Diphenyl-4-Methylphenylamine) (Compound (9)) via Steps A to I.

### (A) Compound (1) 4-Methyltriphenylamine

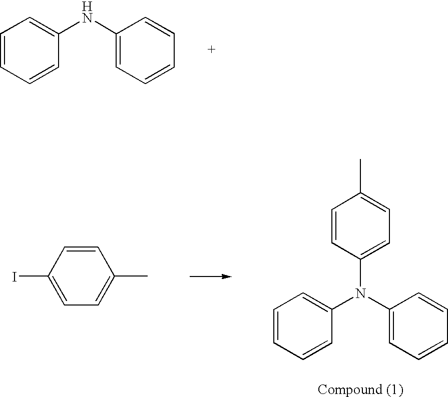

To a flame-dried flask fitted with condenser, nitrogen inlet and outlet, mechanical stirrer and suba-seal was added diphenylamine (50.0 g, 295.5 mmol (1 molar equivalent)), 4-iodotoluene (128.9 g, 591.3 mmol (2 molar equivalents)) and 1,10-phenanthroline (10.7 g, 59.1 mmol (0.2 molar equivalents)). The flask and contents were thoroughly flushed with nitrogen before anhydrous o-xylene (200 ml) was added. The reaction mixture was heated to 110° C. and then copper (I) chloride (5.9 g, 59.1 mmol (0.2 molar equivalents)) and potassium hydroxide (132.6 g, 2364.0 mmol (8 molar equivalents)) were added to the reaction flask. The reaction was heated further with rapid stirring to 150° C. for 24 hours before allowing the reaction to cool to room temperature. Water and dichloromethane (DCM) were added to the solution and this was filtered though celite before extracting the product using DCM. The combined organic fractions were dried over magnesium sulphate (MgSO₄), filtered and concentrated under vacuum. Purification by column chromatography (silica gel 60; 9:1, hexane:DCM) followed by recrystallisation from methanol gave compound (1) as a white solid (46.5 g, 61%), greater than 99% pure by HPLC. ¹H NMR, 300 MHz (CDCl₃) δ 7.24-7.15 (4 H, m, H-Ar), 7.07-6.90 (10 H, m, H-Ar) and 2.29 ppm (3 H, s, Me).

### (B) Compound (2) 4,4′-Dibromo-4″-methyltriphenylamine

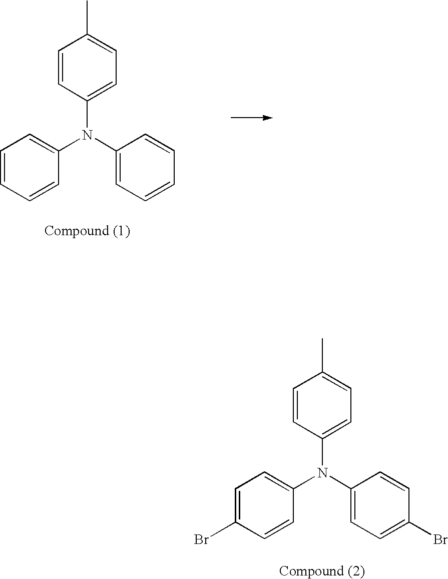

To a flask fitted with nitrogen inlet and outlet, mechanical stirrer and suba-seal was added 4-methyltriarylamine (Compound (1)) (40.0 g, 154.4 mmol (1 molar equivalent)) and N, N-dimethylformamide (DMF) (120 ml). The resulting solution was cooled using dry ice. To the reaction mixture was added a solution of N-bromosuccinimide (NBS) (55.0 g, 308.8 mmol (2 molar equivalents)) dissolved in DMF (280 ml) over 30 minutes, once addition was complete, the dry ice bath was removed. After 1 hour the reaction was complete. The reaction solution was added to water (1 L) and the product extracted three times using hexane. The combined organic fractions were dried over MgSO₄, filtered and concentrated under vacuum. The crude product was crystallised from a mixture of methanol and acetone at 0° C. to yield compound (2) as an off white solid (59.6 g, 93%), 97% pure by HPLC. ¹H NMR, 300 MHz (CDCl₃) δ7.28 (4 H, d, J 8.88 Hz; H-Ar), 7.06 (2 H, d, J 8.22 Hz; H-Ar), 6.94 (2 H, d, J 8.22 Hz; H-Ar), 6.88 (4 H, d, J 8.88 Hz; H-Ar) and 2.29 ppm (3 H, s, Me).

### (C) Compound (3)—Triarylamine diboronic ester

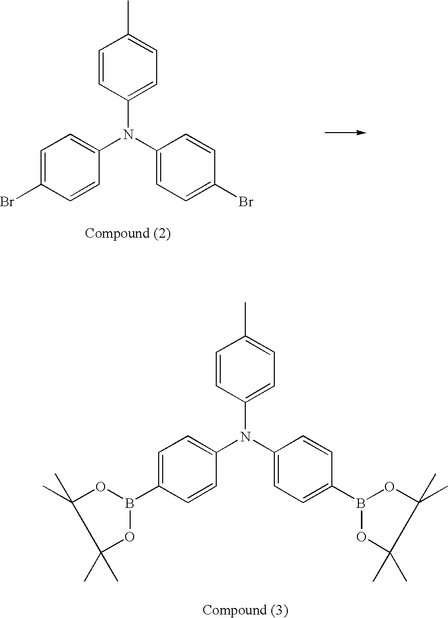

To a flame-dried flask fitted with condenser, nitrogen inlet and outlet, mechanical stirrer and suba-seal was added 4,4′-dibromo-4″-methyltriphenylamine (Compound (2)) (15.0 g, 36.0 mmol (1 molar equivalent)) and the flask flushed with nitrogen for 15 minutes. Anhydrous tetrahydrofuran (THF) (60 ml) was added and the solution cooled to −78° C. using an acetone/dry ice cold bath. N-Butyl lithium(n-BuLi) 2.5 molar (in hexanes) (34.6 ml, 86.4 mmol (2.4 molar equivalents)) was added drop-wise over 30 minutes, and the resulting solution stirred at −78° C. for 1 hour. 2-Isopropoxy-4,4,5,5-tetramethyl-1,3,2-dioxaborolane (19.1 ml, 93.6 mmol (2.6 molar equivalents)) was added and the resulting solution allowed to warm up to room temperature with stirring over night. Water was added to the reaction flask and the product extracted with DCM. The combined organic extracts were dried over magnesium sulphate (MgSO₄), filtered and concentrated under vacuum to give the crude product. Purification by column chromatography (silica gel 60; 9:1, hexane:EtOAc) followed by recrystallisation from methanol gave the title compound as a white solid (12.9 g, 70%), 95% pure by HPLC. ¹H NMR, 300 MHz (CDCl₃) δ 7.66 (4 H, d, J 8.55 Hz; H-Ar), 7.11-6.97 (8 H, m, H-Ar), 2.23 (3 H, s, Me) and 1.33 ppm (24 H, s, boronic ester).

### (D) Compound (4) 4-Methyldiphenylamine

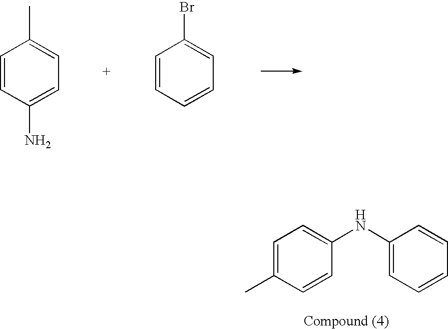

To a flame-dried flask fitted with condenser, nitrogen inlet and outlet, mechanical stirrer and suba-seal was added bromobenzene (26.8 ml, 254.4 mmol (1 molar equivalent)), p-toluidine (30.0 g, 279.8 mmol (1.1 molar equivalents)), racemic-2,2′bis(diphenylphosphino)-1,1′binaphthyl (rac-BINAP) (1.2 g, 1.9 mmol (0.0075 molar equivalents)), tris-(dibenzylidineacetone)dipalladium(0) (Pd₂(dba)₃) (0.6 g, 0.6 mmol (0.0025 molar equivalents)) and sodium tert-butoxide (NaO^{t}Bu) (34.2 g, 356.2 mmol (1.4 molar equivalents)) the flask and contents were then flushed with nitrogen for 15 minutes. Anhydrous toluene (500 ml) was added to the reaction flask and the mixture stirred at 110° C. for 4 hours. On cooling, the reaction mixture was filtered through celite and the toluene solution washed with water, dried over magnesium sulphate (MgSO₄), filtered and concentrated under vacuum. Purification by column chromatography (silica gel 60; 9:1, hexane:DCM) followed by recrystallisation from hexane gave compound (4) as a white solid (32.7 g, 70%). 94% pure by HPLC. ¹H NMR, 300 MHz (CDCl₃) δ 7.29-7.17 (1 H, m, H-Ar), 7.07 (2 H, d, J 8.12 Hz; H-Ar), 7.04-6.97 (4 H, m, H-Ar), 6.88 (1 H, t, J 7.35 Hz; H-Ar), 5.60 (1 H, s, NH) and 2.30 ppm (3 H, s, Me).

### (E) Compound (5) 4-Bromo-4′-methyldiphenylamine

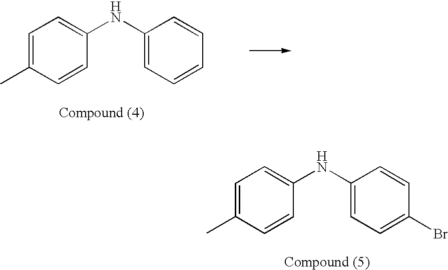

To a flask fitted with nitrogen inlet and outlet, mechanical stirrer and suba-seal was added 4-methyldiphenylamine (Compound (4)) (30.0 g, 163.7 mmol (1 molar equivalent)) and DMF (75 ml). The resulting solution was cooled using dry ice. To the reaction mixture was added a solution of N-bromosuccinimide (NBS) (29.1 g, 163.7 mmol (1 molar equivalent)) dissolved in DMF (75 ml) over 30 minutes. The cold bath was then removed and the reaction allowed to warm to room temperature. After 1 hour the reaction was complete. The reaction solution was added to water (500 ml) and the product extracted three times with hexane. The combined organic fractions were dried over magnesium sulphate (MgSO₄), filtered and concentrated under vacuum. Purification by column chromatography (silica gel 60; 9:1, hexane:DCM) followed by recrystallisation from hexane gave compound (5) as an off white solid (31.8 g, 74%). 96% pure by HPLC. ¹H NMR, 300 MHz (CDCl₃) δ 7.30 (2 H, d, J 8.88 Hz; H-Ar), 7.08 (2 H, d, J 8.00 Hz; H-Ar), 6.96 (2 H, d, J 8.44 Hz; H-Ar), 6.84 (2 H, d, J 8.88 Hz; H-Ar), 5.56 (1 H, s, NH) and 2.30 ppm (3 H, s, Me).

### (F) Compound (6)—tert-butoxycarbonyl(Boc) protected 4-bromo-4′-methyldiphenylamine

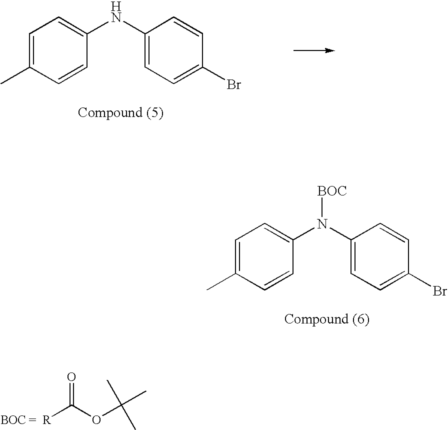

To a flame-dried flask fitted with condenser, nitrogen inlet and outlet, mechanical stirrer and suba-seal was added 4-bromo-4′-methyldiphenylamine (Compound (5)) (3.5 g, 13.4 mmol (1 molar equivalent)), 4-dimethylaminopyridine (DMAP) (0.7 g, 5.4 mmol (0.4 molar equivalents)) and di-^{t}butyl dicarbonate (8.7 g, 40.1 mmol (3 molar equivalents)). The flask contents were then flushed with nitrogen for 30 minutes before anhydrous THF (15 ml) was added. The reaction solution was heated with stirring at 70° C. for 3 hours. The reaction mixture was concentrated under vacuum and the crude material purified by column chromatography (silica gel 60; 8:2, hexane:DCM) followed by recrystallisation from methanol, yielding compound (6) as a white solid (2.4 g, 50%), greater than 99% pure by HPLC. ¹H NMR, 300 MHz (CDCl₃) δ 7.39 (2 H, d, J 8.88 Hz; H-Ar), 7.15-7.01 (4 H, m, H-Ar), 2.33 (3 H, s, Me) and 1.44 (9 H, s, Boc).

### (G) Compound (7) - Di-tert-butoxycarbonyl(boc) protected triarylamine trimer

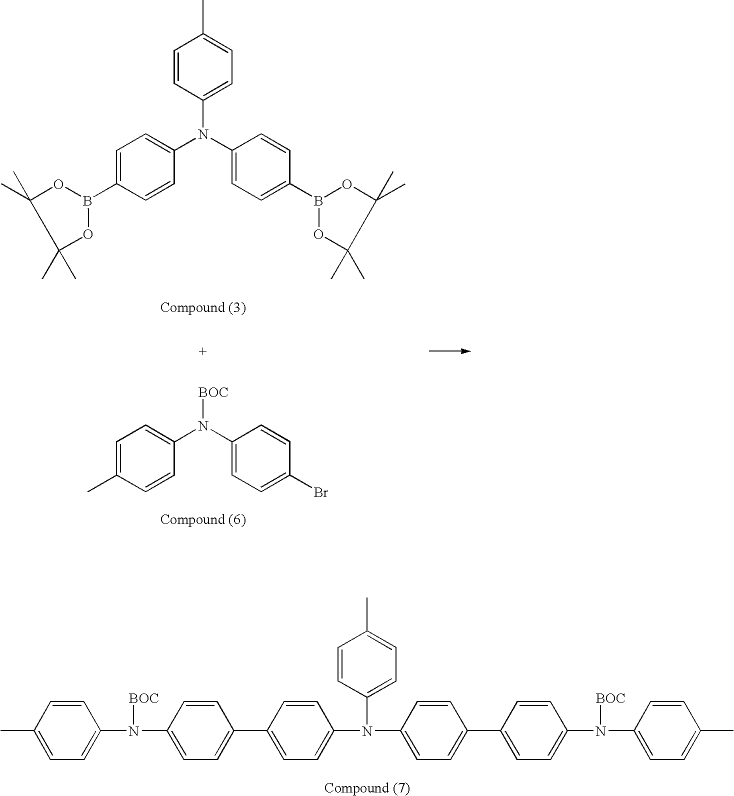

To a flask fitted with condenser, nitrogen inlet and outlet, mechanical stirrer and suba-seal was added compound (3) (10.0 g, 19.6 mmol (1 molar equivalent)), tert-butoxycarbonyl(Boc) protected 4-bromo-4′-methyldiphenylamine (Compound (6)) (17.7 g, 48.9 mmol (2.5 molar equivalents)) and toluene (60 ml). The resulting solution was degassed with nitrogen for 15 minutes and then tetrakis(triphenylphosphine)palladium(0) (Pd(PPh₃)₄) (1.4 g, 1.2 mmol (6 mol %)) was added. To this solution was added degassed 2 M aqueous sodium carbonate (Na₂CO₃) (60 ml) and the reaction mixture rapidly stirred at 110° C. for 24 hours. On cooling, the reaction mixture was diluted with water and the crude product extracted with DCM three times. The combined organic fractions were dried over MgSO₄, filtered and concentrated under vacuum. Purification by column chromatography (silica gel 60; 8:2, hexane:EtOAc) gave compound (7) as a cream/yellow solid (10.3 g, 64%), 91% pure by HPLC. ¹H NMR, 300 MHz (CDCl₃) δ 7.45 (8 H, dd, J 8.55, 6.69 Hz; H-Ar), 7.23 (4 H, m, H-Ar), 7.16-7.04 (16 H, m, H-Ar), 2.33 (9 H, s, H-Me) and 1.46 ppm (18 H, s, H-Boc).

### (H) Compound (8)—Di-NH triarylamine trimer

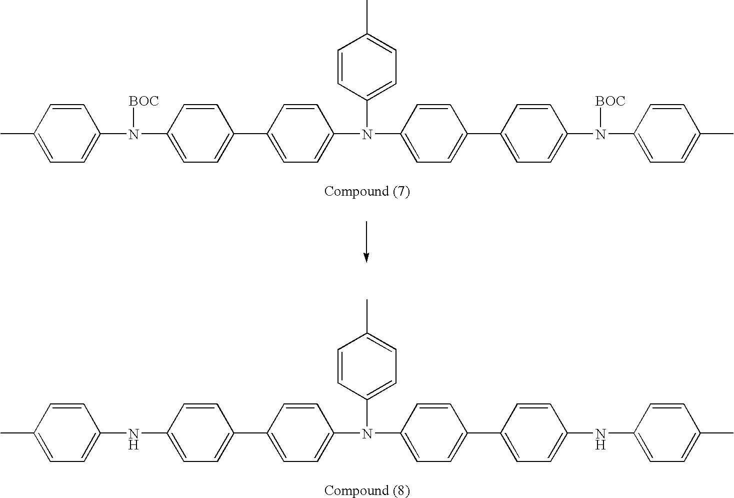

To a flask fitted with nitrogen inlet and outlet, mechanical stirrer and suba-seal was added di-boc protected triarylamine trimer (Compound (7)) (10.3 g, 12.6 mmol) and DCM (20 ml). To this solution, trifluoroacetic acid (TFA) (20 ml) was added and reaction mixture stirred for 1.5 hours. The TFA was neutralised with sodium carbonate (Na₂CO₃) solution and the product extracted with DCM. The combined organic fractions were dried over MgSO₄, filtered and concentrated under vacuum. Purification by column chromatography (silica gel 60; 1:1, hexane:ethyl acetate) followed by a hot methanol wash yielded compound (8) as a yellow/brown solid (6.8 g, 86%), greater than 98% pure by HPLC. ¹H NMR, 300 MHz (D₆ DMSO) δ 8.14 (2 H, s, NH), 7.50 (8 H, m, H-Ar), 7.18-6.95 (20 H, m, H-Ar), 2.28 (3 H, s, H-Me) and 2.23 ppm (6 H, s, H-Me).

### (I) Compound (9)—Cyclo-hexa(diphenyl-4-methylphenylamine)

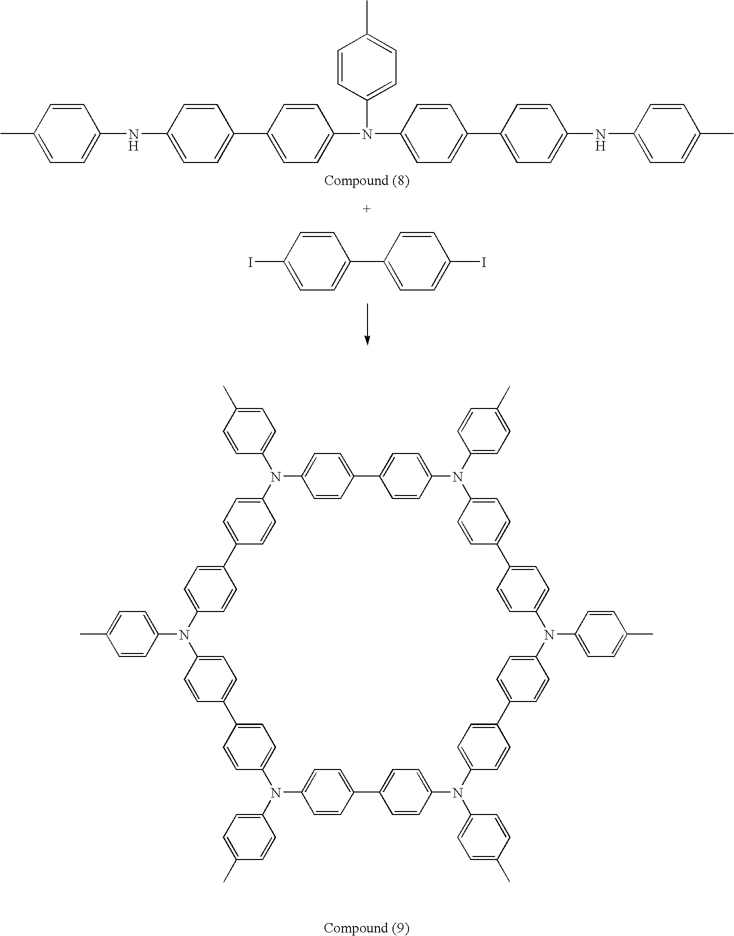

To a flame-dried flask fitted with condenser, nitrogen inlet and outlet, mechanical stirrer, dropping funnel and suba-seal was charged di-NH triarylamine trimer (compound (8)) (2.9 g, 4.9 mmol (0.5 molar equivalents)), 4, 4′-diiodobiphenyl (3.7 g, 9.2 mmol (0.98 molar equivalents)), copper powder 200 mesh (2.4 g, 37.5 mmol (4 molar equivalents)), potassium carbonate (10.6 g, 76.8 mmol (8 molar equivalents)) and 18-crown-6 (0.3 g, 0.9 mmol (0.1 molar equivalents)). The dropping funnel was charged with di-NH triarylamine trimer (compound (8)) (2.9 g, 4.9 mmol (0.5 molar equivalents)). The reaction vessel was flushed with nitrogen for 15 minutes. o-Dichlorobenzene (250 ml) was then charged to the flask and a further o-dichlorobenzene (250 ml) charged to the dropping funnel. The contents of the reaction flask was heated at 180° C. with rapid stirring for 4 hours before the contents of the dropping funnel were added over 1 hour. The resulting mixture was stirred at 180° C. for 14 days. On cooling, the reaction mixture was filtered through celite to remove any insoluble materials, the solution was then concentrated under vacuum, and a dark brown glassy solid resulted. Soxhlet extraction of this solid with acetone removed most of the low molecular weight impurities. The remaining material was dissolved in a minimum amount of refluxing tetrahydrofuran (THF) and allowed to cool to room temperature, during which time a solid precipitated out, which was analysed by HPLC and found to be 70% pure. Purification by column chromatography (silica gel 60; 1:1, toluene:hexane) gave a yellow solid, which by HPLC was 95% pure. Repeated recrystallisations from THF gave compound (9) as white/yellow needles (0.15 g, 2%), greater than 99% pure by HPLC. ¹H NMR, 500 MHz (D₈ THF) δ 7.51 (24 H, d, J 8.43 Hz; H-Ar), 7.15-7.06 (m, 48 H, H-Ar) and 2.34 ppm (18 H, s, Me); ¹³C NMR, 125 MHz (D₈ THF) δ 148.0, 146.0, 135.8, 133.7, 130.7, 128.0, 125.7, 124.7 and 20.9 ppm; m/z (DEI) 152 (50%), 771 (100%); Cyclic voltammetry versus ferrocene, −5.17 eV (reversible) and −5.82 eV (irreversible).

### Example 2

Alternatively, cyclo-hexa(diphenyl-4-methylphenylamine) (Compound (9)) can be prepared from steps A to H as described in example (1) above followed by steps J to M as described below.

### (J) Compound (10) 4-Bromo-4′-(Trimethylsilyl)biphenyl

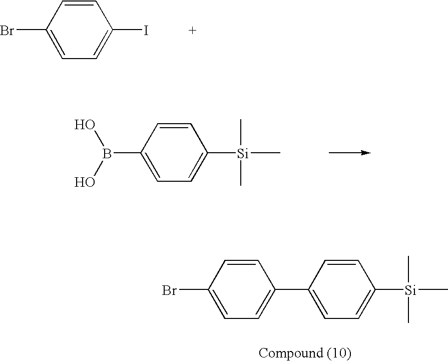

To a solution of 1-bromo-4-iodobenzene (14.14 g, 50 mmol), 4-(trimethylsilyl)phenyl boronic acid (9.71 g, 50 mmol) and Pd(PPh₃)₄ (2.31 g, 2.0 mmol) in degassed 1,4-dioxane (100 ml) was added a solution of potassium carbonate (16.6 g, 120 mmol) in degassed water (50 ml). The mixture was refluxed under a nitrogen atmosphere until the reaction was complete (according to HPLC analysis).

The reaction mixture was then washed with distilled water and brine and then dried over MgSO₄ before being filtered through a silica plug. The crude product was purified by flash column chromatography (silica gel 60; 9:1, hexane:dichloromethane) followed by recrystallisation from methanol to yield a colourless crystalline solid (12.01 g, 78.7%) greater than 99% pure by HPLC. ¹H NMR, 300 MHz (CDCl₃) δ 7.42-7.27 (4 H, AA′BB′, H-Ar), 7.36 (4 H, m, H-Ar) and 0.15 ppm (9 H, s, Si-Me).

### (K) Compound (11)

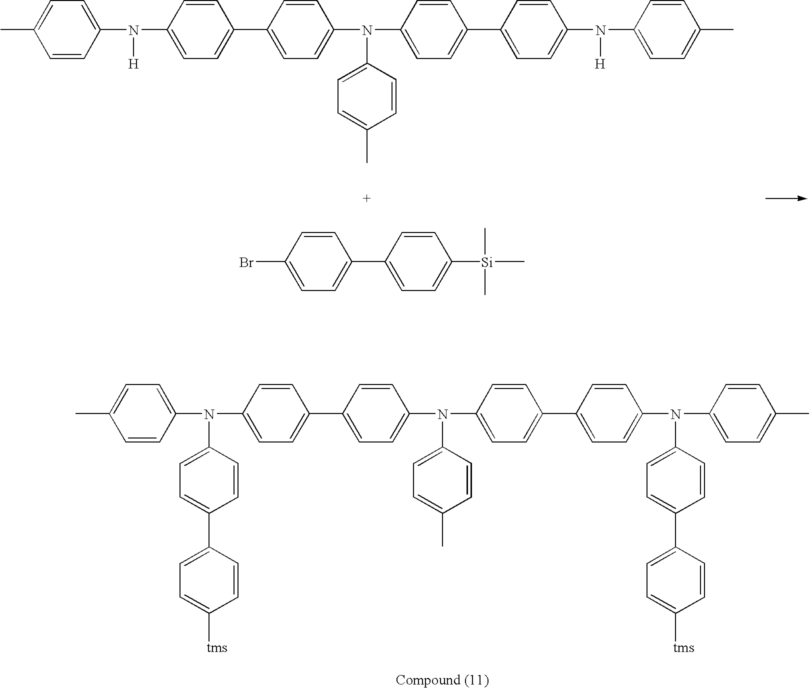

To a solution of compound (8) (1.70 g, 2.74 mmol), 4-bromo-4′-(trimethylsilyl)biphenyl (1.67 g, 5.48 mmol) and sodium t-butoxide (1.58 g, 16.43 mmol) in dry degassed toluene (30 ml) was added a solution of tris(dibenzylidineacetone) dipalladium(O) (Pd₂(dba)₃) (50.1 mg, 0.055 mmol) and 2-(dicyclohexylphosphino)biphenyl (115.7 mg, 0.33 mmol) in dry degassed toluene (5 ml). The mixture was stirred and heated to 100° C. and the progress of the reaction monitored by HPLC. Once the reaction was complete the mixture was cooled to ambient temperature and diluted with toluene (50 ml). The resultant solution was then filtered and the solvent removed in vacuo to yield the crude product, which was then purified by column chromatography (silica gel 60; 95:5, hexane:ethylacetate) followed by recrystallisation from methanol to yield an olive coloured solid (2.66 g, 90.7%) greater than 96% pure by HPLC. ¹H NMR, 300 MHz (CDCl₃) δ 7.65-7.05 (44 H, m, H-Ar), 2.30 (9 H, s, CH₃) and 0.25 ppm (18 H, s, Si-Me).

### (L) Compound (12)

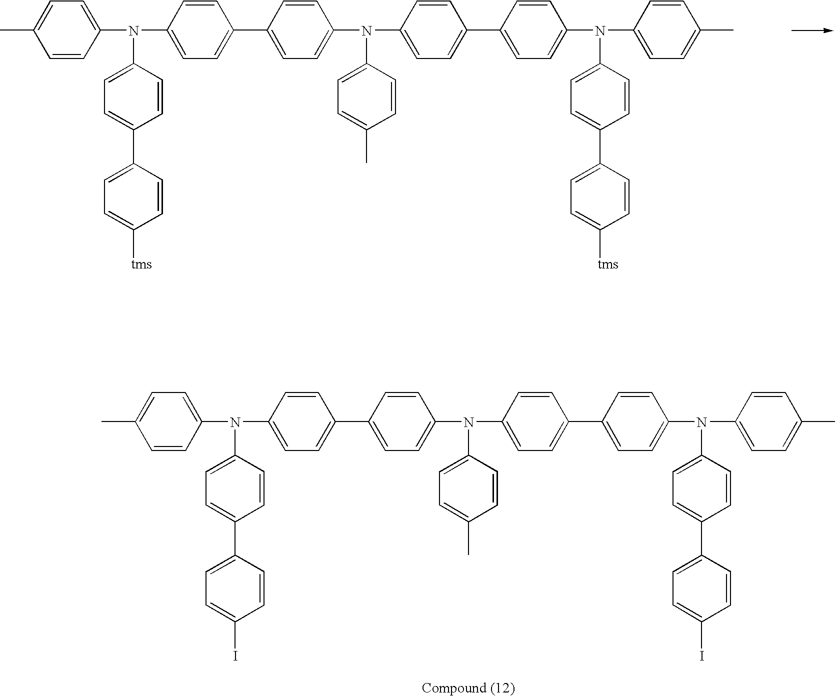

To a stirred solution of (Compound 11) (2.50 g, 2.34 mmol) in dry dichloromethane (16 ml) was added a solution of iodine monochloride (1.14 g, 7.01 mmol) in dry dichloromethane (16 ml) dropwise over a period of 30 minutes at −10° C. The resulting mixture was stirred at this temperature until HPLC analysis indicated that the reaction was complete before a saturated solution of sodium meta bisulphate (Na₂S₂O₅) (17 ml) was added. The organic and aqueous layers were separated and the aqueous layer extracted with dichloromethane (2×50 ml). The combined organic phase was dried (MgSO₄), filtered and concentrated in vacuo. The crude product was purified by flash column chromatography (SiO₂; hexane:ethylacetate, 70:30 (consistency)) followed by recrystallisation from methanol and hexane to yield a tan coloured solid (2.29 g, 86.9%) greater than 95% pure by HPLC. ¹H NMR, 300 MHz (CDCl₃) δ 7.65-7.05 (44 H, m, H-Ar) and 2.30 ppm (9 H, s, CH₃).

### (M) Compound (9)—Cyclohexa(diphenyl-4-methylphenylamine)

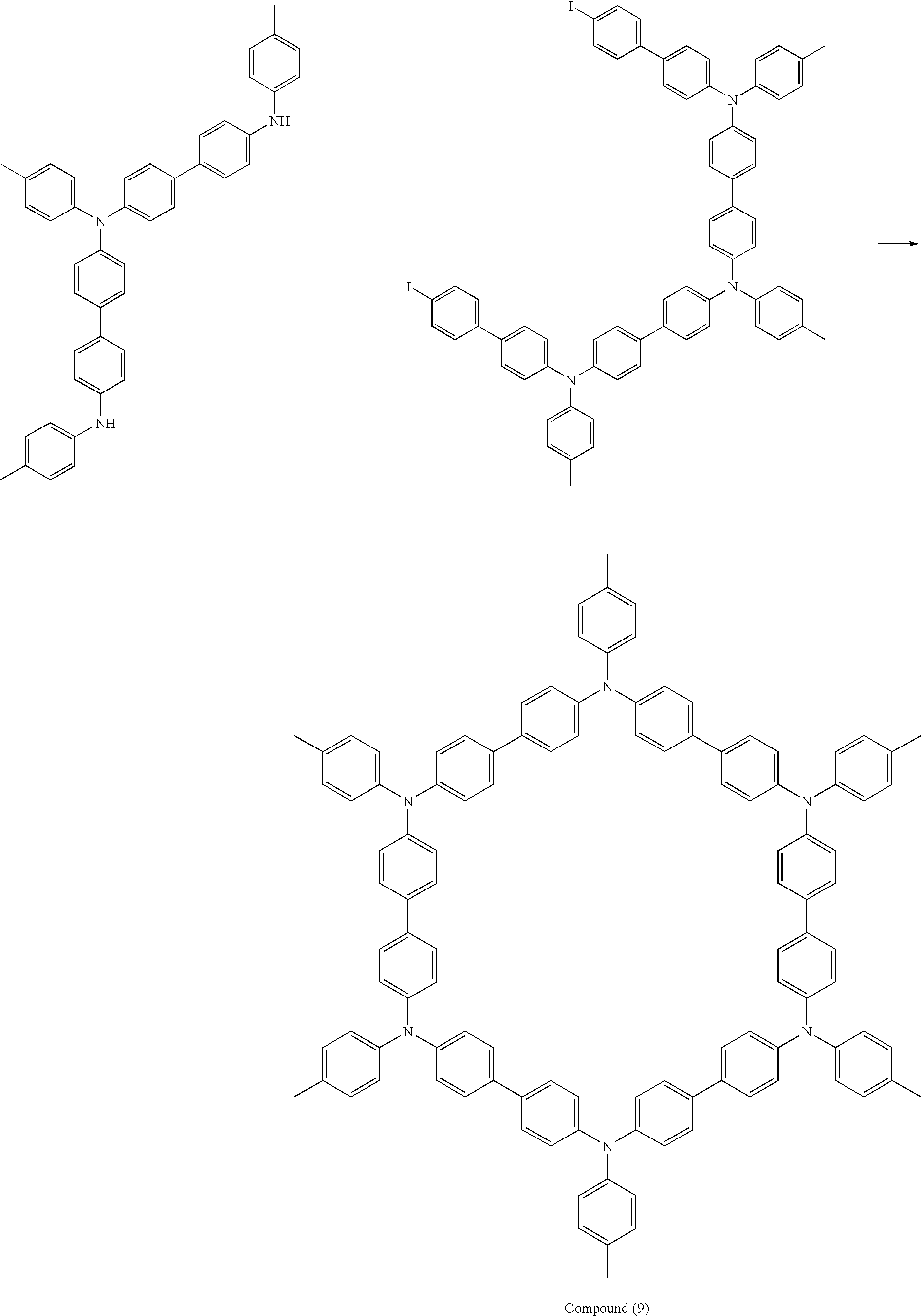

To a solution of Compound (12) (261 mg, 0.221 mmol), Compound (8) (138 mg, 0.221 mmol) and sodium t-butoxide (127.8 mg, 1.33 mmol) in dry degassed o-xylene (6.0 ml) was added a solution of Pd₂(dba)₃ (2.0 mg, 0.0022 mmol) and 2-(dicyclohexylphosphino)biphenyl (4.6 mg, 0.0132 mmol) in dry degassed o-xylene (1 ml). The mixture was stirred and heated to 100° C. and the progress of the reaction monitored by HPLC.

Complete conversion of reactants to the desired product was achieved after 24 hours (as verified by HPLC analysis) and the reaction mixture was cooled to 70° C., diluted with toluene (50 ml) and filtered whilst hot through a bed of celite. The celite layer was subsequently washed further with hot toluene (200 ml). The solvent was removed in vacuo to yield the crude product which was purified by column chromatography (silica gel 60; 50:50; hexane:toluene) and recrystallised from tetrahydrofuran to yield the product as a colourless crystalline solid (150 mg, 44%) greater than 99% pure by HPLC. ¹H NMR, 300 MHz (CDCl₃) δ 7.40 (24 H, m, H-tolyl), 7.10 (48 H, m, H-Ar), 2.35 (18 H, s, CH₃).

### Example 3

Alternatively, compounds (13) and (14) are prepared in analogy to the methods described in example (1) above.

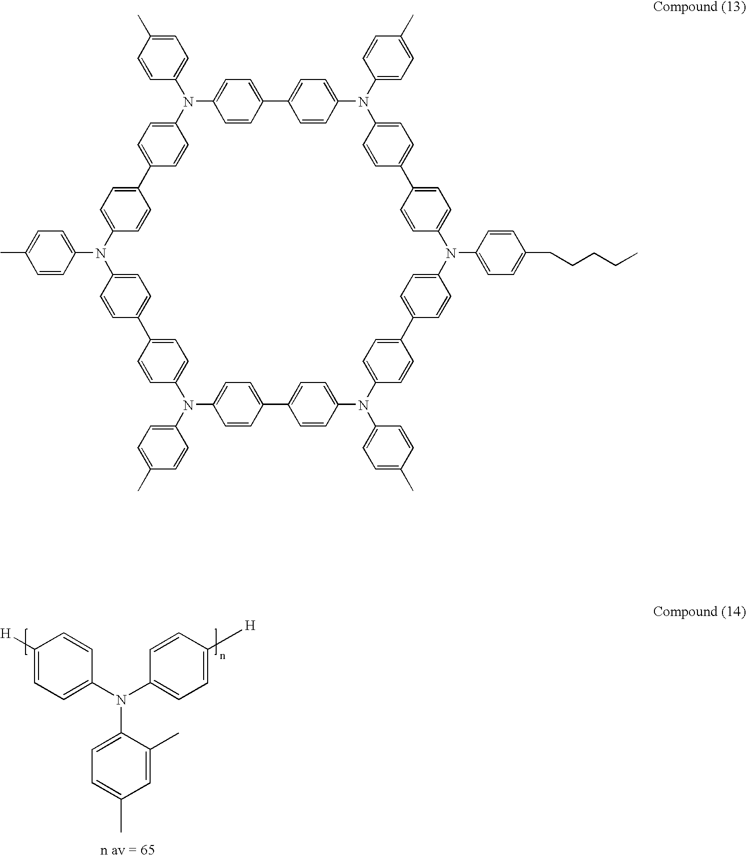

### Determination of the Field Effect Mobility

The field effect mobility of the materials was tested using the techniques described by Holland et al, J. Appl. Phys. Vol.75, p.7954 (1994).

In the following examples a test field effect transistor (FET) was manufactured by using a PEN poly(ethylene-2,6-naphthalene dicarboxylate) substrate upon which were patterned Pt/Pd source and drain electrodes by standard techniques, for example, shadow masking. A semiconductor formulation was made using a cyclic triarylamine compound for example compound (9) blended with an inert binder resin (poly(alpha-methyl styrene)(p-αMS) Aldrich catalogue number 19,184-1.

The semiconductor formulation was dissolved one part into 99 parts of toluene, and spin coated onto the substrate at 1000 rpm for 20 seconds to yield a thin film of less than 100 nm. To ensure complete drying the sample was placed in an oven for 20 minutes at 100° C. A solution of an insulator material for example TOPAS™8007 (dielectric constant=2.2-2.3) was then spin coated onto the semiconductor giving a thickness typically in the range of 0.5 to 1 μm. The sample was placed once more in an oven at 100° C. to evaporate solvent from the insulator. A gold gate contact was defined over the device channel area by evaporation through a shadow mask. To determine the capacitance of the insulator layer a number of devices were prepared which consisted of a non-patterned Pt/Pd base layer, an insulator layer prepared in the same way as that on the FET device, and a top electrode of known geometry. The capacitance was measured using a hand-held multimeter, connected to the metal either side of the insulator. Other defining parameters of the transistor are the length of the drain and source electrodes facing each other (W=30 mm) and their distance from each other (L=130 μm).

The voltages applied to the transistor are relative to the potential of the source electrode. In the case of a p-type gate material, when a negative potential is applied to the gate, positive charge carriers (holes) are accumulated in the semiconductor on the other side of the gate insulator. (For an n channel FET, positive voltages are applied). This is called the accumulation mode. The capacitance/area of the gate dielectric Cᵢ determines the amount of the charge thus induced. When a negative potential V_{DS} is applied to the drain, the accumulated carriers yield a source-drain current I_{DS} which depends primarily on the density of accumulated carriers and, importantly, their mobility in the source-drain channel. Geometric factors such as the drain and source electrode configuration, size and distance also affect the current. Typically a range of gate and drain voltages are scanned during the study of the device. The source-drain current is described by Equation 1:

$${I}_{DS} = \frac{μ {WC}_{i}}{L} ( ( {V}_{G} - {V}_{0} ) {V}_{DS} - \frac{{V}_{DS}^{2}}{2} ) + {I}_{Ω} \qquad Equation 1$$

where;

V₀ is an offset voltage, and I_{Ω} is an ohmic current independent of the gate voltage and is due to the finite conductivity of the material. The other parameters have been described above.

For the electrical measurements the transistor sample was mounted in a sample holder. Microprobe connections were made to the gate, drain and source electrodes using Karl Suss PH100 miniature probe-heads. These were linked to a Hewlett-Packard 4155B parameter analyser. The drain voltage was set to −5 V and the gate voltage was scanned from +20 to −60V in 1 V steps. In accumulation, when |V_{G}|>|V_{DS}| the source-drain current varies linearly with V_{G}. Thus the field effect mobility μ, can be calculated from the gradient (S) of I_{DS} versus V_{G} given by Equation 2.

$$S = \frac{μ {WC}_{i} {V}_{DS}}{L} \qquad Equation 2$$

All field effect mobilities quoted below were calculated using this regime (unless stated otherwise). Where the field effect mobility varied with gate voltage, the value was taken as the highest level reached in the regime where |V_{G}|>|_{DS}| in accumulation mode. The results show the charge mobility obtained when the cyclic triarylamine compounds of the present invention were tested in combination with up to 50% by weight of resin.

In accordance with the present invention, an FET device prepared with a 50:50 percent by weight composition of compound 9 with resin poly α-methyl styrene, gave a mobility μ=3.26×10⁻⁴cm²V⁻¹s⁻¹.

### Use Example 1

For this purpose compound (9) is dissolved with poly α-methyl styrene at 0.5% solids in toluene. The solution is spin coated onto masked Pt/Pd patterned source/drain electrodes. A 5% solution of Topas 8007 in methyl cyclohexane was used as the gate insulator. Compound (9), gave a mobility, μ=3.26×10⁻⁴cm²V⁻¹s⁻¹. Average I_{On/Off}=3600.

### Use Example 2

For this purpose compound (13) is dissolved with poly α-methyl styrene at 1% solids in toluene. The solution is spin coated onto masked Pt/Pd patterned source/drain electrodes. Cytop was used as the gate insulator. Compound (13), gave a mobility, μ=2.5×10⁻⁴cm²V⁻¹s⁻¹. Average I_{On/Off}=18,500.

### Use Example 3

For this purpose compound (9) is dissolved with compound (14) (1:1 by weight) at 0.5% solids in toluene. The resulting solution is then spin coated upon masked Pt/Pd patterned source/drain electrodes. Topas is used as the gate insulator. Compound (9) gives an average mobility, μ of 2.9×10⁻³ cm²/Vs. Average I_{On/Off}=18,100.

1. A compound of Formula (1): 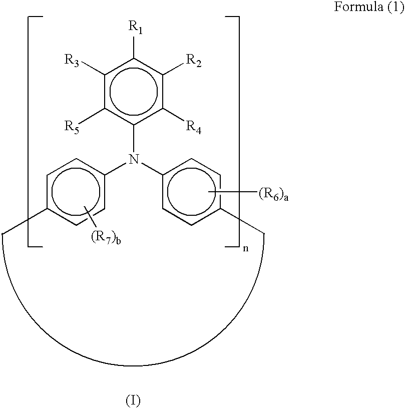
wherein:
each of R₁, R₂, R₃, R₄, R₅, R₆ and R₇ which may be the same or different on each triarylamine unit of Formula (1) and the same or different from one triarylamine unit of Formula (1) to another is independently hydrogen or an optionally substituted substituent;
n is from 5 to 20; and
a and b are each independently 0,1, 2, 3 or 4.

2. A compound according to claim 1 wherein each of R₁, R₂, R₃, R₄ and R₅ independently comprises hydrogen or a substituent selected from the group comprising an optionally substituted C₁-C₄₀ carbyl or hydrocarbyl group; an optionally substituted C₁-C₄₀ alkoxy group; an optionally substituted C₆-C₄₀ aryloxy group; an optionally substituted C₇-C₄₀ alkylaryloxy group; an optionally substituted C₂-C₄₀ alkoxycarbonyl group; an optionally substituted C₇-C₄₀ aryloxycarbonyl group; a cyano group (—CN); a carbamoyl group (—C(═O)NH₂); a haloformyl group (—C(═O)—X, wherein X represents a halogen atom); a formyl group (—C(═O)—H); an isocyano group; an isocyanate group; a thiocyanate group; a thioisocyanate group; an optionally substituted amino group; a hydroxy group; a nitro group; a CF₃ group; a halo group; an optionally substituted silyl group; and
each of R₆ and R₇ is independently selected from the group comprising H, CH₃, F, CN or CF₃.

3. A compound according to claim 1 wherein:
n is 6 to 15.

4. A compound according to claim 1 wherein R₁, R₂, R₃, R₄ and R₅ are each independently a saturated, linear or branched acyclic group.

5. A compound according to claim 5 wherein the saturated, linear or branched acyclic group comprises an optionally substituted C₁₋₄₀ alkyl group, more preferably an optionally substituted C₁₋₂₀ alkyl group, most preferably an optionally substituted C₁₋₁₂ alkyl group.

6. A compound according to claim 1 wherein:
R₆ and R₇ are each independently H or —CH₃.

7. A compound according to claim 1 wherein n=6.

8. A compound according to claim 1 which comprises a field effect mobility μ of more than 10⁻⁴cm²V⁻¹s⁻¹, more preferably more than 10⁻⁴cm²V⁻¹ s⁻¹.

9. A composition comprising:
(i) a cyclic triarylamine compound of Formula (1) according to claim 1; and
(ii) a synthetic organic polymer resin, wherein the resin may be a semiconductor.

10. A composition according to claim 9 comprising between 10 and 90 weight % of cyclic triarylamine compound and 90 to 10 weight % of synthetic organic polymer resin.

11. A composition according to claim 9 in the form of fibres, films or sheets.

12. A composition according to claim 9 wherein the synthetic organic polymer resin has a permittivity at 1000 Hz of no higher than 3.3 and greater than 1.7.

13. A composition according to claim 9 wherein the synthetic organic polymer resin preferably has a number average molecular weight (Mn) of between 1×10³ and 1×10⁶, more preferably at least 3000, most preferably at least 5000.

14. A composition according to claim 9 wherein the synthetic organic polymer resin comprises a copolymer.

15. A composition according to claim 9 which further comprises one or more of the following:
a plasticiser, an antistatic agent, a colorant, a dopant, a surfactant and/or a reinforcing material.

16. A composition according to claim 9, which further comprises one or more solvents.

17. A composition according to claim 9 wherein the proportions of resin to cyclic triarylamine compound comprises 1:99 to 99:1, more preferably 20:1 to 1:20 and most preferably 2:1 to 1:2.

18. Use of the composition as claimed in claim 9 in an electronic device.

19. Use of the composition as claimed in claim 9 in an electrophotographic apparatus.

20. An organic semiconducting layer for use in an electronic device comprising the composition as claimed in claim 9.

21. A layer as claimed in claim 20 wherein the layer is deposited on a part of an electronic device by solution coating.

22. A layer as claimed in claim 20 comprising a charge mobility μ of greater than 10⁻⁴cm²V⁻¹s⁻¹.

23. A layer as claimed in claim 20 wherein the layer is prepared by
(i) depositing on a substrate for an electronic device a liquid layer comprising a composition; and
(ii) forming from the liquid layer a solid layer.

24. A layer as claimed in claim 20 wherein the layer is deposited on a part of an electronic device by one of the following coating or printing techniques:
dip coating, spin coating, ink jet printing (including continuous and drop-on-demand and fired by piezo or thermal processes), letter press printing, screen printing, doctor blade coating, roller printing, reverse-roller printing, offset lithography printing (including photolithographic processes), flexographic printing, web printing, spray coating, brush coating, pad printing, bar coating or gravure coating.

25. A layer as claimed in claim 20 wherein the layer is used as a semiconducting layer in one of the following electronic devices: field effect transistor (FET), organic light emitting diode (OLED), photodetector, chemical detector, photovoltaic cell capacitor sensor, logic circuit, display, or memory device.

26. A FET, OLED, photodetector, chemical detector, photovoltaic cell capacitor sensor, logic circuit, display, or memory device comprising a compound, composition or layer as claimed in claim 1.

27. A process for preparing a compound as claimed in claim 1 comprising the steps of:
(i) preparing a triarylamine monomer from a suitably substituted aniline or secondary amine and a suitably substituted aryl halide;
(ii) brominating available para position on the triarylamine monomer;
(iii) converting the brominated monomer to a diboronic ester following by quenching;
(iv) synthesising a secondary amine from a suitably substituted aniline and a suitably substituted aryl halide;
(v) brominating the secondary amine in an available para position;
(vi) protecting the mono-brominated secondary amine with a protecting group;
(vii) coupling together the compounds produced from steps (iii) and (vi);
(viii) removing the protecting groups; and
(ix) cyclising the resultant diamine with for example an iodine substituted biophenyl compound to produce a cyclic triarylamine compound,
wherein steps (i) to (iii) can be performed before or after steps (iv) to (vi).

28. A process as claimed in claim 27 wherein step (ix) is replaced by the following steps:
(x) preparing a substituted biphenyl compound, substituted for example with bromine and trimethyl silane;
(xi) preparing a triarylamine trimer by the reaction of a suitably substituted bisamine trimer and the substituted biphenyl compound from step (x);
(xii) preparing an iodo functionalised compound by replacing the trimethylsilyl group with iodine; followed by
(xiii) cycling the iodo compound from step (xii) with suitably substituted bis amine compound to yield a cyclic triarylamine compound.
